(19) United States
(12) Patent Application Publication
(10) Pub. No.: US 2009/0012820 A1
(43) Pub. Date: Jan. 8, 2009

(54) Medication dose administration and inventory management

(76) Inventors: Shane Bishop, Kelowna (CA); Theodore Ritchie, Lake Country (CA); Marina Andricevic, Kelowna (CA)

Correspondence Address: BRINKS HOFER GILSON & LIONE, P.O. BOX 10395, CHICAGO, IL, 60610, US

(21) Appl. No.: 12/006,099

(22) Filed: Dec. 28, 2007

(30) Foreign Application Priority Data
Jul. 6, 2007 (CA) 2,593,174

(51) Int. Cl. G06Q 50/00

(52) U.S. Cl. 705/3

(57) ABSTRACT

A system for managing administration of prescribed medications to patients is disclosed. The system includes a server for receiving prescription fill requests which are based on prescriptions for the prescribed medications, and for causing uniquely identifiable medication packages to be produced based on the prescription fill requests and in conformity with the prescriptions. Each medication package contains a single dose of at least one of the prescribed medications. The server is further for receiving and maintaining medication package records specifying the contents of each particular medication package. The system includes an interface for providing access to the prescriptions and the medication package records for validating administration of the prescribed medications to the patients. Each single dose of prescribed medication is uniquely identifiable by identifying the medication package containing that single dose and by accessing the medication package records specifying the contents of that medication package.

### COPYRIGHT NOTICE

A portion of the disclosure of this patent document contains material that is subject to copyright protection. The copyright owner has no objection to the facsimile reproduction by anyone of the patent document or the patent disclosure, but otherwise reserves all copyrights whatsoever.

### BACKGROUND OF THE INVENTION

1. Field of the Invention

The invention relates generally to computerized inventory management systems and more particularly to systems for managing medication administration and inventory.

2. Description of the Related Art

In long-term healthcare facilities, patients are typically prescribed medications by physicians and the medications are administered periodically by caregivers over an extended period of time. Typically, the prescriptions are not filled at the facility itself, but are forwarded to a pharmacy for fulfillment. While pharmacies traditionally filled prescriptions manually (e.g. by filling bottles and applying labels to the bottles), modern pharmacies commonly employ automated procedures and equipment designed to reduce opportunities for human error inherent in the manual preparation of medication packages.

One such common improvement is the use of blister cards in which all the medications for a particular patient are laid out in individual bubbles on the card. As compared to the traditional pill bottle, blister cards make it much easier for a caregiver or patient to see what medications are to be taken and when. Although blister cards are capable of greatly improving prescription compliance at the time of administration compared to bottles, typical blister cells are often too small to accommodate all medications required for a particular administration event (i.e. dosing date and time), and there remains an opportunity for human error in selecting the correct blister or blisters to be administered.

A considerable increase in safety has been achieved by the introduction of automation at the pharmacy, particularly in the form of automated strip-packaging devices such as the PACMED™ automated dose packager offered by companies such as McKesson APS™ and Parata Systems™. One such system is illustrated in FIG. 1. With such systems, strips of bags may be produced wherein each bag contains individual doses of medication for a particular patient and dosing time. Each bag is connected to the adjacent bags by a perforated section thereby allowing the end bag to be readily torn off. The strips are produced in order of scheduled administration and each bag is imprinted with the dosing details for its contents. Because the medications are in a strip, if all of the medications scheduled for a particular dosing time cannot fit into a single bag, they will simply flow over into a second or third sequential bag. Consequently, by using such strips it is less likely that a medication will be missed at any given dosing time as compared to bottles or blister packs thereby improving prescription compliance.

However, the reliable production of convenient medication packages is only one half of the equation. In order to optimize safety and prescription compliance, any medication administration system which tracks the administration of individual doses must cooperate with such medication packages so as to ensure that the right medication is given to the right patient at the right time.

In this regard, many facilities use traditional medication administration records (“MARs”) to monitor medication administration. A typical manual MAR is reproduced in FIG. 2. A MAR is simply a chart outlining all of the medications a patient is scheduled to receive and indicates dosing times. It is used as a record which may be cross-checked to ensure that no medications have been missed. At the point of care, caregivers are supposed to record the medications administered and time of administration along with other pertinent information.

Manual MARs suffer from certain drawbacks, however, including limited space for recording the requisite information resulting in a record that is difficult to read leading to a loss of information. Manual MARs must also be manually updated, by caregivers, with any changes to a patient's medication regime (e.g. specific starting and ending times for any new prescriptions). Furthermore, while manual MARs provide a passive reference for checking that all medications have been administered, they do not provide any mechanism for actively informing the caregiver that a dose has been missed.

In some long-term healthcare facilities, paper MARs are being replaced by computer systems including electronic medication administration records (“eMARs”). A basic eMAR is merely an electronic version of a paper MAR. While some functions can be automated, the overall functionality of an eMAR remains essentially the same as a paper MAR. Both continue to present opportunities for human error, as the accuracy of either system depends substantially on the attention of the caregiver in recording administration details into the MAR or eMAR, as the case may be. Furthermore, once a medication has been exhausted, separate processes for refilling medication inventory must be followed introducing further opportunities for human error.

In the procedures of typical facilities, prescriptions are entered into the facility's eMAR before the prescriptions are filled by the pharmacy. Once the prescriptions have been filled by the pharmacy, the eMAR is used to record the administration of the prescribed medications. Inasmuch as such systems are used to record patient prescriptions and display such prescriptions to the caregiver at the point of care, they are useful for indicating what medication ought to be administered to any particular patient.

However, neither a paper MAR nor an eMAR provides means for reliably confirming that any particular dose of medication to be administered to a patient is correct and consistent with the recorded prescriptions. Even in cases where medications are packaged using automated systems as described above, the task of ensuring prescription compliance continues to be performed substantially by the caregivers who administer the medications, resulting in unavoidable opportunities for human error.

In order to address the risk of human error at the point of care, some facilities employ pharmacies which use automated packagers to fill prescriptions, as described above, in cooperation with the facility's MAR or eMAR system. In such cases, the facility's eMAR and the pharmacy system will both contain the same prescription information which will be used by the pharmacy system to direct the automatic packaging of medications. However, in known systems, the medication packages ultimately received by the facility typically do not precisely match the prescriptions upon which they are based. For example, some pharmacy systems do not generally accommodate for the beginning or ending of a prescription partway through a day; such systems can only direct an automated packager to generate packages based on whole numbers of days. If, for example, a prescription directs the administration of a medication twice daily at breakfast and supper from Monday until Friday, but the prescription is filled on Monday afternoon, the pharmacy system will also send Monday morning's dose which would be incorrectly filled since that dosing time has already elapsed.

In order to compensate for this problem, known pharmacy systems and automated packagers employ what is commonly called a ‘filter’ which may reside in the pharmacy system, at the automated packager or both. The ‘filter’ receives the prescription information from the pharmacy system and ‘filters off’ any unwanted doses. Unfortunately, this method creates an inconsistency between the underlying prescription and medication packages actually produced. The medication ultimately received by the facility will therefore not correspond precisely to the underlying prescription. In addition, there are numerous other reasons that medication packages generated by an automated packager would not correspond precisely to the underlying prescription, including errors during the packaging process.

While this sort of inconsistency is not ordinarily a problem in most facilities, as pharmacies will generally send enough medication in any event and caregivers will know what medication to give by reference to the prescription, the inconsistency between the underlying prescription and the medication actually received renders individual-dose tracking of the medication impractical. Thus, it is not generally possible in known medication administration systems to track individual doses of medication from packaging to the point of care. The confirmation of ‘what’ is administered remains in the judgement of facility dispensaries and caregivers and is therefore subject to human error.

Furthermore, since the medication ultimately received by the facility may not correspond precisely to the medication prescribed, the exact start and end times of administration are usually based on the medication on hand rather than what was actually prescribed. Prescription compliance is therefore susceptible to flaws in the inventory management of the facility.

Various systems have been proposed in the art but do not overcome the above-described challenges. Examples of such proposals include United States Patent Application Publication No. 2005/0261940 by Gay et al, United States Patent Application Publication No. 2003/0200726 by Rast, United States Patent Application Publication No. 2005/0131733 by Lubow, and U.S. Pat. No. 6,021,392 to Lester et al., the entire contents of each of which is incorporated herein by reference. Although these references disclose desirable aspects, none of them disclose alone or in combination a medication administration management system for easily and accurately tracking patient-specific individual-dose/multiple-medication packages from the point of packaging to the point of care.

Thus, in order to maximize safety (i.e. minimize errors in the administration of medications) and prescription compliance, a medication administration management system must be able to easily and accurately track individual doses of medication from the point of packaging to the point of care. This requires that a MAR or eMAR system incorporate complete information as to precisely how a given prescription has been filled, with the ability to associate every individual dose of medication to a specific patient and a specific dosing time.

### BRIEF SUMMARY OF THE INVENTION

The above-described advantages are provided by the systems and methods described hereinafter. In accordance with the invention, every individual dose of medication administered to a patient is verified as being correct prior to administration. In order to accomplish this, every individual dose of medication is tracked from the time of packaging to the point of care on a patient-specific basis.

The invention is found in a system, as follows, for managing administration of prescribed medications to patients. The system includes a server for receiving prescription fill requests which are based on prescriptions for the prescribed medications, and for causing uniquely identifiable medication packages to be produced based on the prescription fill requests and in conformity with the prescriptions. Each medication package contains a single dose of at least one of the prescribed medications. The server is further for receiving and maintaining medication package records specifying the contents of each particular medication package. The system includes an interface for providing access to the prescriptions and the medication package records for validating administration of the prescribed medications to the patients. Each single dose of prescribed medication is uniquely identifiable by identifying the medication package containing that single dose and by accessing the medication package records specifying the contents of that medication package.

The invention is also found in a system, as follows, for tracking a dose of a medication and for validating an administration of the dose of the medication to a patient. The system has a module for receiving prescription information and a data record, each associated with the dose of the medication. The data record is further associated with a unique package ID uniquely identifying a medication package containing the dose of the medication and no other dose of the medication. The system further has a database for receiving and storing the data record in association with the unique package ID, and for receiving and storing the prescription information. The system also has an interface for providing access to the database for accessing the data record and the prescription information for validating the administration of the dose of the medication to the patient. The data record is accessed by reference to the unique package ID.

The invention is also found in a system, as follows, for producing a uniquely identifiable medication package containing a single dose of a prescribed medication. The system has an interface for receiving a prescription fill request which is based on a prescription for the prescribed medication. The system also has a processor for conforming the prescription fill request to the prescription thereby producing a filtered fill request, and for causing the medication package to be produced in accordance with the filtered fill request, wherein the medication package is uniquely identifiable by a unique package ID. The system has an interface for receiving data regarding the single dose of the prescribed medication, the data being associated with the unique package ID of the medication package. The system also has a database for storing the data.

The invention is also found in a method, as follows, for uniquely identifying a single dose of a prescribed medication for validating an administration of the single dose to a patient. A prescription fill request is received; the prescription fill request is based on a prescription for the prescribed medication. The prescription fill request is conformed to the prescription thereby producing a filtered fill request. A uniquely identifiable medication package is caused to be produced in accordance with the filtered fill request. The medication package contains, with respect to the prescribed medication, exclusively the single dose of the prescribed medication, wherein the medication package is uniquely identifiable by a unique package ID. Data regarding the single dose is received, the data being associated with the unique package ID of the medication package. The prescription and the data are stored, the data being stored in association with the unique package ID. Access is provided to the prescription and the data, wherein the data is accessed by reference to the unique package ID. The single dose of prescribed medication is thereby uniquely identified, for validating the administration of the single dose to the patient.

The invention is also found in a computer program product for enabling a computer to uniquely identify a single dose of a prescribed medication for validating an administration of the single dose to a patient, as follows. The computer program product includes software instructions for enabling the computer to perform predetermined operations, and a computer readable medium bearing the software instructions. The predetermined operations including the following steps. A prescription fill request is received which is based on a prescription for the prescribed medication. The prescription fill request is conformed to the prescription thereby producing a filtered fill request. A uniquely identifiable medication package is caused to be produced in accordance with the filtered fill request; the medication package contains, with respect to the prescribed medication, exclusively the single dose of the prescribed medication; the medication package is uniquely identifiable by a unique package ID. Data regarding the single dose is received, and is associated with the unique package ID of the medication package. The prescription and the data are stored, with the data being stored in association with the unique package ID. Access is provided to the prescription and the data, wherein the data is accessed by reference to the unique package ID. The single dose of prescribed medication is thereby uniquely identified, for validating the administration of the single dose to the patient.

### BRIEF DESCRIPTION OF THE DRAWINGS

An understanding of the exemplary embodiments will be obtained from the following description, with reference to the following drawings in which:

FIG. 1 illustrates a prior art medication packager and prior art medication packages;

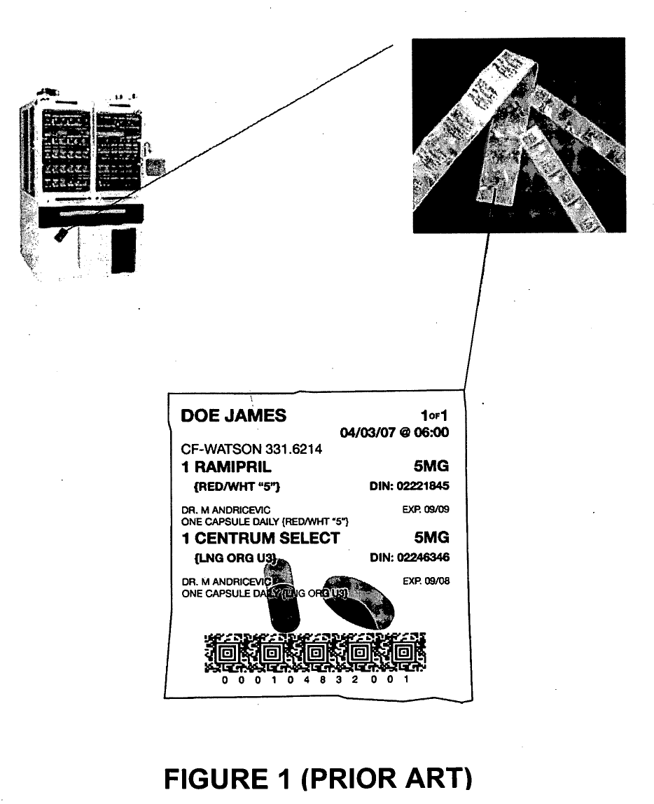

FIG. 2 shows a prior art manual MAR;

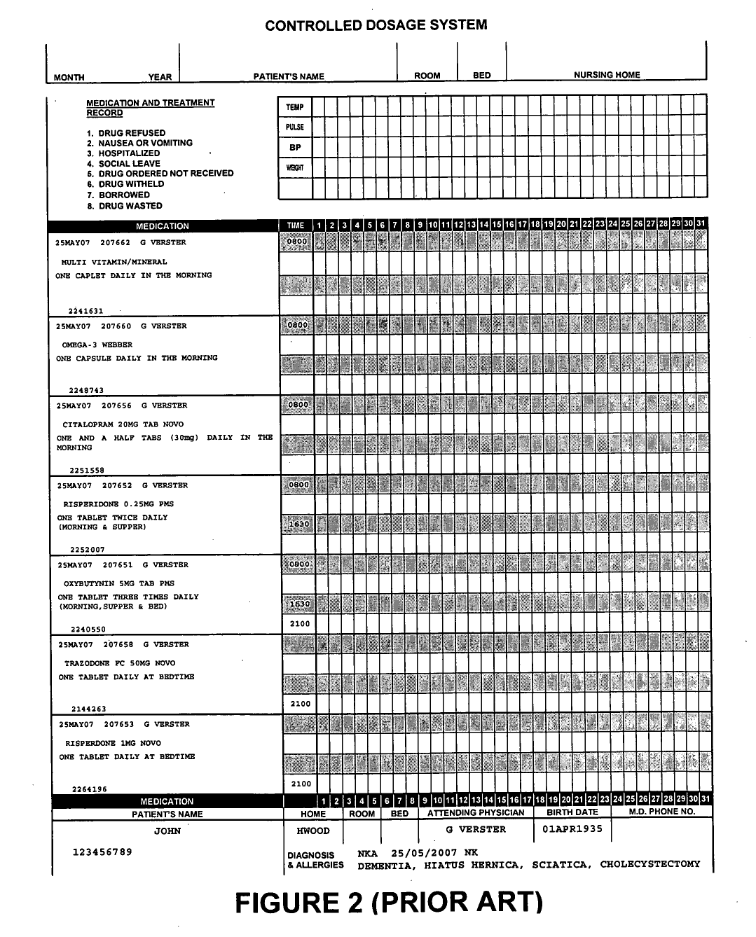

FIGS. 3A & 3B show exemplary medication packages bearing human-readable information and machine-readable barcodes, with FIG. 3A showing a linear barcode and FIG. 3B showing a 2D barcode;

FIG. 4 shows an exemplary unique package ID illustrating its various components;

FIG. 5 shows a flowchart illustrating a method of producing uniquely identifiable medication packages containing individual doses of prescribed medications;

FIG. 6 shows a schematic diagram of an exemplary system for producing uniquely identifiable medication packages containing individual doses of prescribed medications, and for managing administration of the prescribed medications to patients;

FIG. 7 shows a flowchart illustrating a method of managing an inventory of uniquely identifiable medication packages, and of validating administration of medication doses contained in such packages to patients;

FIGS. 8A-8P show screenshots of the interfaces of exemplary systems described herein, wherein FIG. 8A shows a patient (resident) identification/search interface;

FIG. 8B shows a patient homepage; or main; interface;

FIG. 8C shows the interface of FIG. 8B overlaid with a dialogue box including medication reminders;

FIG. 8D shows a Daily MAR interface;

FIG. 8E shows a Daily TAR interface;

FIG. 8F shows the interface of FIG. 8D overlaid with a dialogue including an image of medications associated with a selected medication package, and means for recording actions undertaking in connection with such medications;

FIG. 8G shows an interface for recording and reviewing the administration of fluid medications to selected body areas;

FIG. 8H shows an interface for placing a medication on hold;

FIG. 8I shows a dialogue box for informing a caregiver that a scanned medication package does not correspond to the presently-selected patient, and for selectively proceeding to another patient;

FIG. 8J shows a Monthly MAR interface;

FIG. 8K shows an interface for entering and reviewing scheduled actions and reminders;

FIG. 8L shows an interface for entering and reviewing patient vitals;

FIG. 8M shows an interface for entering and reviewing patient consults;

FIG. 8N shows an interface for selecting reports;

FIG. 8O shows an interface for submitting a medication refill request to a pharmacy; and

FIG. 8P shows the interface of FIG. 8O overlaid with a dialogue box indicating that a requested medication refill has already been submitted.

Where appropriate, the same reference numerals are used in the drawings to indicate like features in all of the drawings.

### DETAILED DESCRIPTION OF EXEMPLARY EMBODIMENTS OF THE INVENTION

Embodiments of the invention are useful for managing the inventory and point-of-care administration of patient-specific, individual-dose/multiple medication packages which are trackable throughout their entire journey from packaging to administration. The exemplary system described hereinafter is particularly advantageous in facilities having numerous patients requiring the regular administration of multiple medications over an extended period of time.

The exemplary system is described hereinafter as including a number of modules and components, each providing certain functionality and interfacing with other modules, components, and systems in providing such functionality. Persons skilled in the art will be able to conceive of other embodiments wherein the functionality is divided differently between different modules and components, or included in a single module or component, or wherein different interfaces are employed. Persons skilled in the art will also be able to conceive of embodiments wherein various modules and components, or functionality, are distributed amongst and executed by various separate platforms or alternatively a single platform. It is to be appreciated that such variants do not depart from the invention but are merely alternative embodiments.

In the exemplary embodiment, the system resides on one or more servers having a processor, a memory, storage, and interfaces operating in connection with a suitable operating system, such as Microsoft™ Windows 2003™, which is operatively connected to a network such as the Internet. The system is interfaced through the network, using any suitable web browser, by means of any suitable web server such as Microsoft™ Information Server 6.0™.

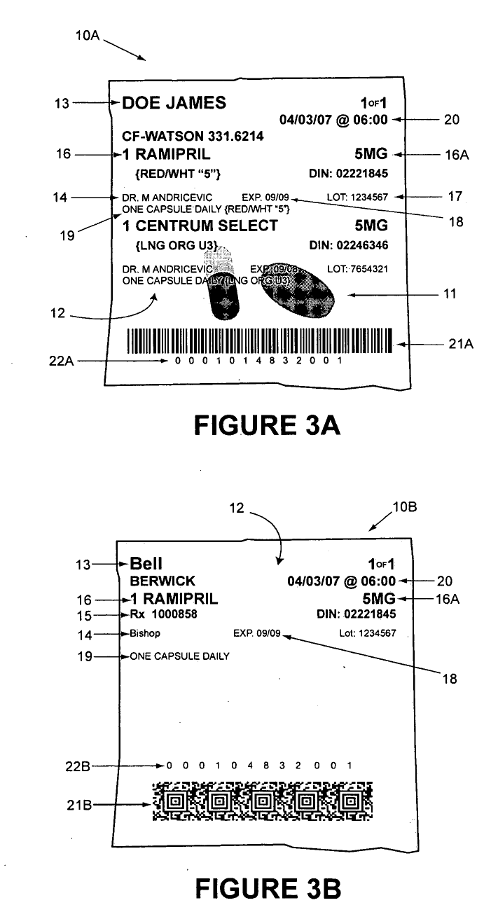

The exemplary system cooperates with one or more automated medication packagers to produce packages such as packages 10A & 10B, as shown in FIGS. 3A & 3B, respectively, each containing, as shown in FIG. 3A, an individual dose of preferably all of the medications 11 (which are capable of processing by an automated packager) prescribed for the patient. While any known automated packager may be employed, the exemplary embodiment employs an automated packager such as the PACMED™ automated packager sold by McKession APS™ and produced by Parata Systems™ which produces connected strips of bags perforated between each pair of bags to allow for the easy tearing-off of the end bag in the strip for dispensing of the medications contained therein. As shown in FIGS. 3A & 3B, each bag is also imprinted by the automated packager or otherwise with human-readable information 12 including patient 13 and physician identities 14, prescription information 15 (e.g. prescription number), contained medication details 16 including individual dose size 16A, slot numbers 17 and expiry dates 18, administration instructions 19 (including dosing date and time 20), and a barcode 21A, 21B embodying a unique package ID 22A, 22B, as described further hereinafter. The strips of bags are typically arranged in a roll for easy handling.

Generally, each such medication package preferably contains all of the medications capable of packaging which are directed to a specific patient for a particular administration event (e.g. dosing date and time). The contained medications may correspond to more than one prescription. In situations where the package size is insufficient to contain all doses of medication required for a particular administration event, multiple adjacent packages may be used. Medications not capable of packaging by the automated packager are also managed by the system as described hereinafter.

In accordance with the invention, every package of medication is associated with a specific patient, prescription, dosing date/time, and all other pertinent information by means of a unique package ID. This association enables the tracking of individual doses of medication from packaging to administration, to enforce proper administration of the medication, and to prevent misadministration. In the exemplary system, the unique package ID is also embodied in a barcode applied to the medication package to facilitate cooperation of the unique package ID with the system.

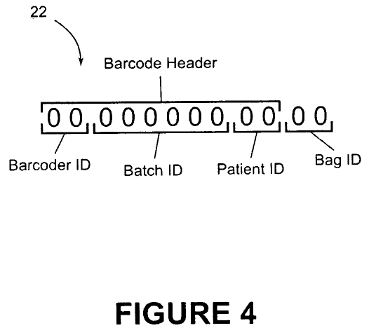

Although any means for uniquely identifying each medication package may be employed, the exemplary embodiment as shown in FIG. 3B employs a 12-character ID 22B, and corresponding barcode 21B, as a unique package ID. With reference to FIG. 4, the unique package ID 22 is generally illustrated, and has the following components: a barcoder ID, a batch ID, a patient ID, and a bag ID. The barcoder ID uniquely identifies the system which has generated the package ID: since there will typically be a number of systems generating unique package IDs at any given time, the barcoder ID ensures that no two such systems will ever generate the same unique package ID. The batch ID is a sequential counter of the batches of medication packages which the particular system has produced. The patient ID identifies the patient to receive the medication within that batch as each batch may contain medications for multiple patients. Finally, the bag ID identifies the specific package in the set of packages created in this batch for this patient, and may therefore also be viewed as identifying the particular dose of medications contained in the package. If multiple packages are needed to contain all of the doses to be administered at a particular time, then the bag ID for each package may be viewed as identifying the partial dose of medications for the dosing time.

As illustrated in FIG. 4, the unique package ID without the bag ID may be treated as a barcode header and used to readily identify a related group of medication packages for the purposes of inventory management. In addition, the barcode header may be used to identify pre-packaged medications containing multiple doses that cannot be readily segmented into individual doses, as described hereinafter.

The characters of the unique package ID in the exemplary embodiment are alpha-numeric. Accordingly, there are 36¹²≈4.7×10¹⁸ possible package IDs, including nearly 1300 possible patient IDs and as many packages in the batch. Thus, for practically all facilities this form of unique package ID will be sufficient to uniquely identify every medication package needed. If an alternative form of unique package ID is preferable in a given context, such may be used and remain within the scope of the invention. While any alternative form of unique package ID, including alternative characters, may be employed, it is preferred that any such unique package ID be compact, as typical medication packages ordinarily provide a limited amount of space for the application of the unique package ID and barcode along with the human-readable information. The barcode encoding the unique package ID and printed on each medication package may comprise any suitable encoding scheme and graphical representation including a linear barcode 21A, as shown in FIG. 3A, or a 2D barcode 21B (e.g. Aztec Code™), as shown in FIG. 3B.

In the exemplary embodiment, the unique package ID does not itself encode the associated information (e.g. patient and physician identities, prescription information): it serves only as a means to associate the two (i.e. it encodes only the unique package ID). While in other embodiments the barcode may additionally encode the information as well as serving as a unique identifier, it is preferable that the unique package ID encode as little information as possible (e.g. only enough to provide the above-indicated association) as a barcode encoding less information is generally more reliably read by scanning devices than a barcode containing more information. In addition, such a unique package ID is more practical in cases where the barcode is unreadable and the ID must be entered manually.

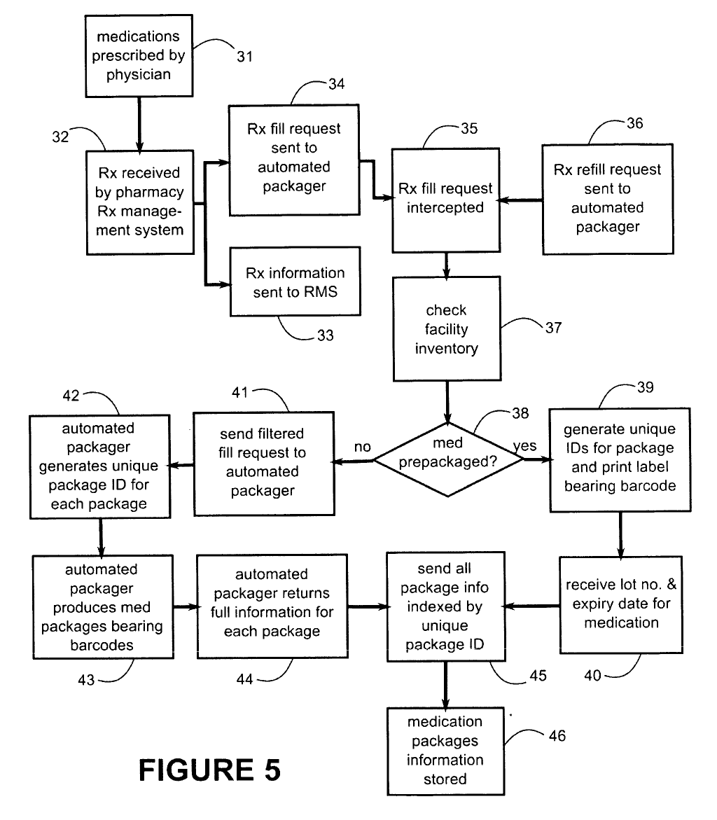

As indicated above, the exemplary embodiment is particularly useful in a long-term healthcare facility or prison in which a caregiver administers medications to resident patients. With reference to FIGS. 5 & 6, a typical medication lifecycle begins with a physician in the facility prescribing medications for a specific one of the patients (step 31). The prescription is received by a pharmacy 51 and is entered into the pharmacy's prescription management system 52 which is ordinarily a computer system connected to a network (step 32). The prescription management system preferably operates a prescription order service through which remote systems may securely interface through a network in order to submit prescription orders. An example of such a service is the E-Prescribing™ service offered by SureScripts™ in the United States. Accordingly, in the exemplary system, the pharmacy system additionally or alternatively receives prescription information from a resident management system 54 (also described hereinafter) interfacing the prescription management system 52 through a network connection 53. Where the pharmacy system does not have such capability, prescriptions are typically received manually, by telefax, or by telephone and entered into the pharmacy system manually. Generally, any appropriate means of communication of prescriptions to the pharmacy may be employed.

Having received prescription information, the prescription management system 52 generates a prescription fill request generally intended to be sent to an automated packager 56 which includes a controller system (step 34). The prescription management system 52 also transmits the patient, physician, medication, and prescription information to the resident management system module (“RMS”) 54 (step 33). Based on the prescription fill request, the controller system of the automated packager 56 is generally configured to generate packaging instructions which cause the automated packager 56 to produce medication packages (or labels, in the case of pre-packaged medications) in accordance with the prescription fill request. The packaging instructions include all necessary information for filling the packages as well as for imprinting on the packages: e.g., patient and physician identities, medications, dosages, and administration instructions.

A filter application module 57 is operatively connected between the prescription management system 52 and the automated packager 56 via network connections 55, 60 to intercept prescription fill requests which would otherwise be sent directly to the automated packager 56 (step 35). A prescription fill request so intercepted may also be a prescription refill request sent by the prescription management system 52 (step 36).

The prescription fill request intercepted by the filter application includes in the exemplary embodiment at least the following information, though it is to be appreciated that any desired information can be included:
the patient's identity (e.g. Mr. John Smith);
the prescription number (e.g. Rx # 1234);
the medication name (e.g. Tylenol™);
a mnemonic identifying the medication;
the dosing times (e.g. 08:00 and 16:00);
individual dose quantity (e.g. take 2);
administration directions (e.g. take two pills twice daily on a full stomach); and the prescribing physician's identity (e.g. Dr. John Carter).

Upon intercepting a prescription fill or refill request, the filter application 57 checks for existing inventory at the facility in respect of the medications specified in the prescription fill or refill request, as the case may be (step 37). This feature is further discussed hereinafter.

The filter application 57 determines, in respect of each prescribed medication requiring filling, whether the medication is pre-packaged (e.g. a bottle containing a fluid, an inhaler) or requires packaging by the automated packager (e.g. pills generally received by the pharmacy in bulk lots) (step 38).

If the medication to be filled is pre-packaged, the filter application 57 generates a unique package ID for the package (step 39). In this regard, each instance of the filter application module 57 has a unique barcoder ID, as discussed above. The filter application 57 causes a label-making means, e.g. a label printer workstation 58 connected to the filter application module 57 via a network connection 58A to create a label for application to the package (step 39). Alternatively, and as discussed above, the automated packager 56 may also be configured to produce such labels if desired. In any event, the label so produced bears the unique package ID and a bar code embodying the unique package ID assigned to the package as well as human-readable information. The filter application 57 receives lot number and expiry date information for each medication package from an operator or otherwise (step 40).

If the medication is not pre-packaged (e.g. pills), and therefore requires packaging by the automated packager 56, then the filter application 57 transmits a filtered fill request to the automated packager 56 (step 41). The filtering function of the filter application module 57 may be any suitable process whereby the fill request received from the pharmacy prescription management system 52 is conformed to the actual prescription. This may include adjusting or otherwise altering the fill request so as to precisely reflect the underlying prescription. For example, if the received fill request directs the filling of doses exceeding the prescription period, perhaps due to an inability in the prescription management system 52 to issue fill requests for partial days, then the filter application 57 filters off such additional doses exceeding the prescription so that only doses precisely matching the prescription period are filled.

In the exemplary system, the automated packager 56 produces a strip of bags each preferably containing an individual dose of at least one of the prescribed medications. As discussed above, it is preferable that each medication package so produced contain an individual dose of every prescribed medication to be administered at a given dosing date and time, thereby requiring the caregiver to open and verify only one medication package. However, if the size of the medication packages is insufficient to contain all of the doses to be administered at a particular dosing date and time, then further sequential packages may be produced.

The automated packager 56 generates a unique package ID for each medication package so produced (step 42) and produces and prints on each package the unique package ID, a barcode encoding the unique package ID, as well as human-readable information received from the filter application 57 relating to the contained doses (e.g. patient and physician identity, medications, the prescription(s), and administration instructions) (step 43). The automated packager 56 also prints on each bag each contained medication's lot number and expiry date (received by the packager 56 when it was loaded with the medications). The automated packager 56 returns to the filter application 57 all information regarding each medication package, including uniquely identifying codes received from the filter application 57 as well as the medication lot numbers and expiry dates of the contained medications, associated with the unique package ID of the package (step 44).

In addition, if the automated packager 56 itself filters off doses, resulting from some internal functionality of the packager 56, then the information returned to the filter application 57 will accurately reflect such additional filtering. In any event, the filter application module 57 will have complete and accurate information for every medication package produced by the automated packager 56 regardless of any steps deviating from the original fill request transmitted by the pharmacy prescription management system 52.

In the exemplary embodiment, therefore, the unique package IDs of medication packages produced by the automated packager 56 are generated by the packager 56, whereas the unique package IDs of pre-packaged medications are generated by the filter application 57. In any event, a unique package ID is generated for each and every medication package, whether it is pre-packaged or produced by an automated packager 56.

In other embodiments, the unique package ID is, in every case, generated by the filter application. However, having the automated packager generate the unique package IDs for packages it produces provides a number of advantages. In cases where a single dosing time's medications must be spread over a number of medication packages, the automated packager is well disposed to track which doses are placed in each of a set of packages, and pass this information back to the filter application. Furthermore, if a mechanical failure of the automated packager occurs during a packaging operation, the automated packager is well disposed to track which medications in the filtered fill request have been packaged and which have not; if the entire fill request is restarted, then a portion of the fill request will be duplicated, and an operator of the packager can select which of the two versions will be forwarded to the facility, and therefore which corresponding set of data records should be forwarded to the filter application. Furthermore, if the packager has performed any other filtering operation, for any reason, the information returned to the filter application will nevertheless accurately characterize the medication packages which were produced.

Thus, for each batch of medication packages produced, the filter application will have complete and accurate information for each and every medication package, indexed by unique medication by ID, thereby enabling the reliable tracking of every individual dose of medication. When this information is forwarded to the facility for medication administration management and inventory tracking, as described hereinafter, the facility can be confident that it accurately represents the contents of the medication packages. Furthermore, the above-described process ensures that each batch of medication packages accurately fulfills all of the prescriptions for each patient including correct dosing times.

The filter application may further be configured to adjust the prescription fill request in order to accommodate administration instructions received from the physician or facility. For example, if some of the medications prescribed to a patient may be taken by the patient him- or herself, while the remaining medications must be administered by a caregiver, the filter application may adjust the fill request so as to package the self-administered medications separately from the caregiver-administered medications. Once packaged, one strip of medications would then go to the patient, while the remaining strips would be delivered to the facility. For example, all of the medication packages may be delivered to the facility, and the packages containing self-administered medications may be delivered to the patient, while the remaining medications are retained by the facility and administered by caregivers as described hereinafter.

Once all of the information for a batch of medication packages, each uniquely identifiable by unique package ID, has been received by the filter application 57, it is then forwarded to the RMS module 54 of the system (step 45), introduced hereinabove, which has already received the patient, physician, medication, and prescription information from the pharmacy prescription management system. For this purpose and others, the filter application module 57 is operatively connected to the RMS module 54 by a network connection 61. Alternatively, the filter application 57 and RMS 54 modules reside on the same server, and the connection 61 consists of internal logical connections between the modules.

The RMS 54 includes a database 62 and an electronic medication administration record (“eMAR”) 63. The RMS 54 further includes any components as are necessary or desirable for carrying out its functionality, as described herein. Although persons skilled in the art will be able to conceive of alternative embodiments, the exemplary embodiment employs a suitable database server such as Microsoft™ SQL Server 2005™ to access the RMS database 62. Alternatively, an object-relational mapping solution such as nHibernate™ may be used.

The information associated with each package received from the filter application 57 is stored in the RMS database 62 (step 46). This information may take any form, including data records arranged or organized in any suitable manner, and indexed by unique package ID and/or otherwise. In general, any organization of information wherein all of the information pertaining to each medication package is uniquely associable with the unique package ID of that package is suitable. In the exemplary system, each record is indexed by a unique dose ID for each dose of medication, and is related to the patient, physician, medication and prescription information received from the pharmacy prescription management system. Each dose record is also associated with the unique package ID of the corresponding medication package, and further contains all of the information related to the dose received from the filter application 57 along with other desired information.

The RMS database 62, therefore, contains a record corresponding to each dose of medication containing all relevant information including, but not limited to: patient and physician identities; prescription number; medication name; date and time for administration; dose quantity; administration instructions; and lot number and expiry date of the medication. Since the record is also associated with the unique package ID of the medication package containing the dose, each unique package ID identifies all of the dose records corresponding to the doses contained in the medication package; since each medication package contains at most one individual dose of each prescribed medication, the unique package ID therefore uniquely identifies an individual dose of a prescribed medication contained in that medication package.

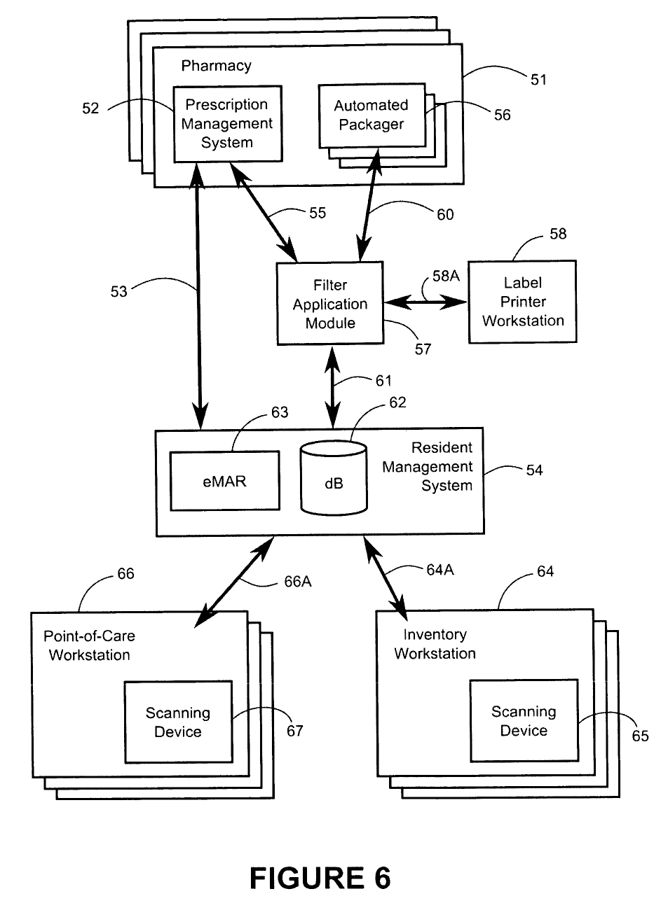

As is illustrated generally in FIG. 6, the filter application 57 may interface with the prescription management system 52 of any number of pharmacies 51, and with any number of automated packagers 56 residing in or associated with any number of pharmacies 51. In addition, any automated packager 56 may alternatively be independent and not associated with any particular pharmacy.

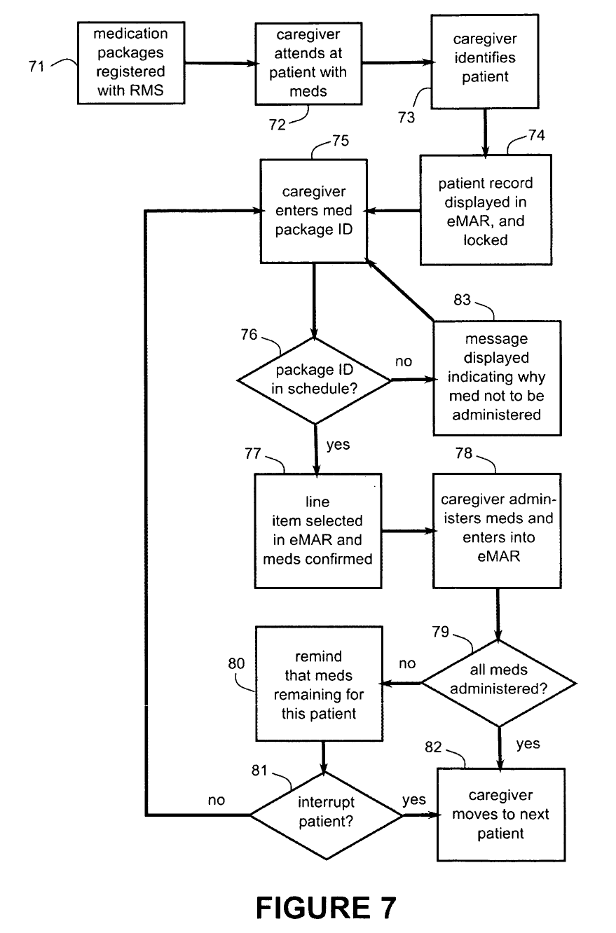

With reference to FIG. 7, batches of medication packages received by the facility are registered with RMS (step 71) before the packages are forwarded for administration to the facility's patients. Accordingly, with reference to FIG. 6, an inventory workstation 64 is operatively connected to the RMS 54 via a network connection 64A and preferably includes a scanning device 65 for scanning the unique package ID barcodes imprinted on the medication packages. Thus, any particular medication package may be registered with the RMS 54 by scanning the package's barcode. The database record indexed by the unique package ID embodied in the barcode is updated to indicate that this package has been received in the facility's inventory.

When the facility receives a batch of packages for a particular patient (e.g. a roll of bags), the entire batch may be registered with RMS at once by scanning the barcode on any one of the packages and indicating to the system that the entire batch of packages having the barcode header (see above) of the scanned barcode is being received. Thus, a facility may quickly and easily register with RMS the receipt of medication packages into its inventory. As described above, the barcode header is also useful for identifying pre-packaged medications containing multiple doses which are not easily segmented into individual doses. In either case, all of the RMS database records associated with the barcode header are updated to register receipt of the packages into the facility's inventory.

Once medication packages are received into a facility's inventory and registered with RMS, they may be distributed to the appropriate locations within the facility in accordance with the facility's practice. In the case of a roll of bags intended for a specific patient, the roll may be placed, for example, in a secure container near the patient's bed or room so that a caregiver may access the roll of bags at the time of administration. If the packages are individually dispensed from a dispensary in the facility, then each dose required for multiple patients in a round may be collected together for a caregiver to transport to each bedside.

In any event, and with reference to FIG. 7, when it is time for a caregiver's medication rounds, the caregiver attends at each patient in the round with the medication packages for that patient and that date and time (step 72). Administration of the medications is carried out using the RMS 54 which includes an interface accessible, e.g. by a point-of-care workstation 66 used by the caregiver via a network connection 66A. A scanning device 67 is preferably operatively connected to the workstation for scanning the package barcodes. Workstations 66 may be provided at convenient locations in the facility, or the caregiver may have a cart bearing a workstation 66 and scanning device 67, as well as containers holding the medication packages needed for the round.

Each point-of-care workstation may be of any form suitable to carry out the functions described herein. It may be a microcomputer, including a laptop or tablet, running a web browser which provides access to the RMS server by any suitable means including a wireless LAN network operatively connected to the RMS server through the Internet. Alternatively, each workstation may be a handheld device, or any other technology adapted to be advantageous in the circumstances of the user. Generally, any technological implementation which performs the functions described herein may be used.

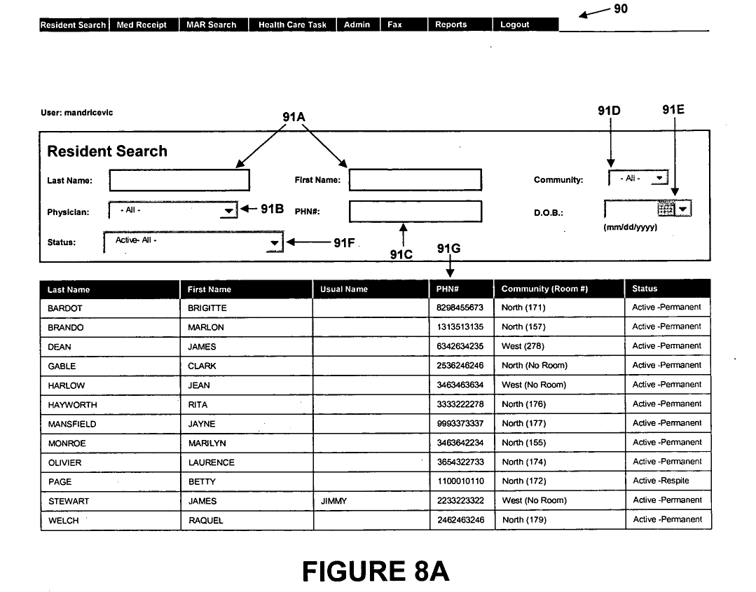

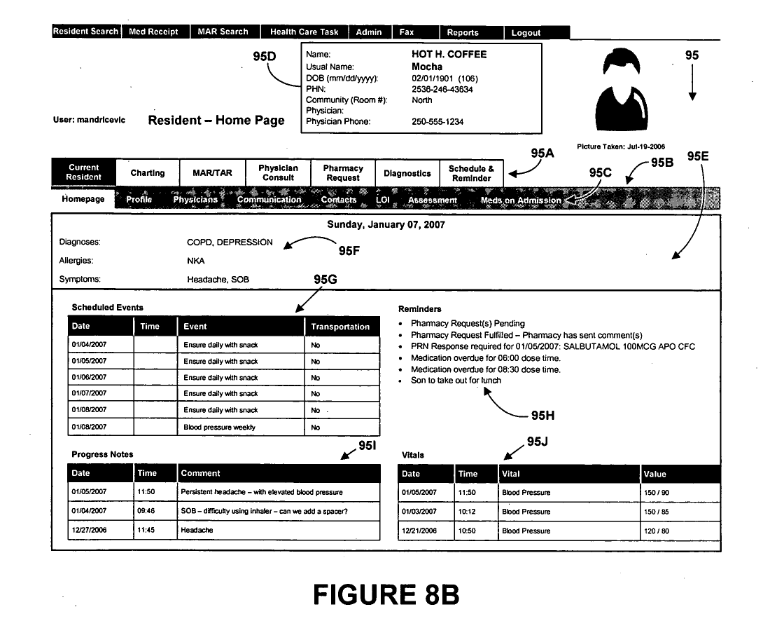

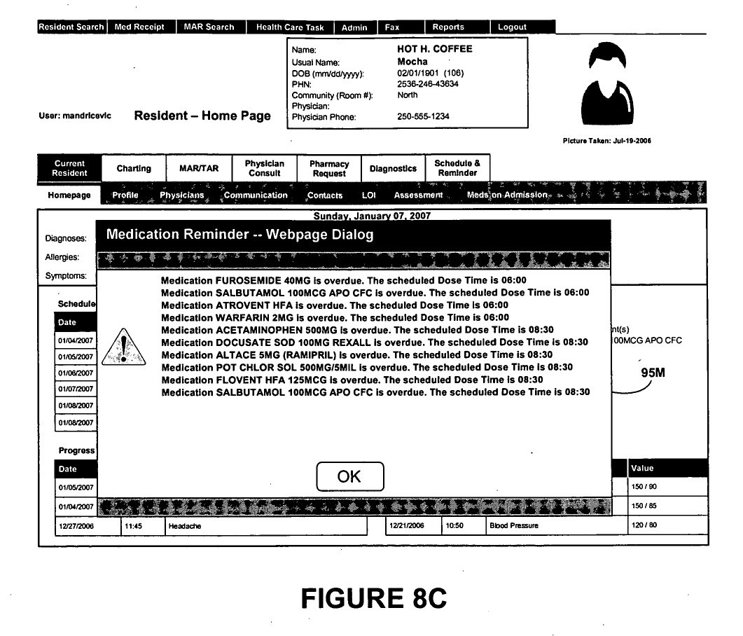

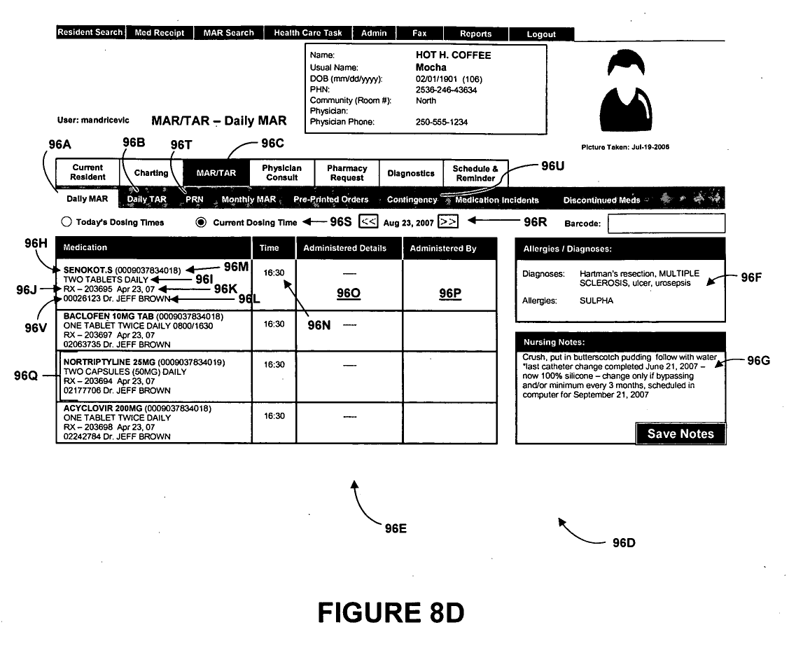

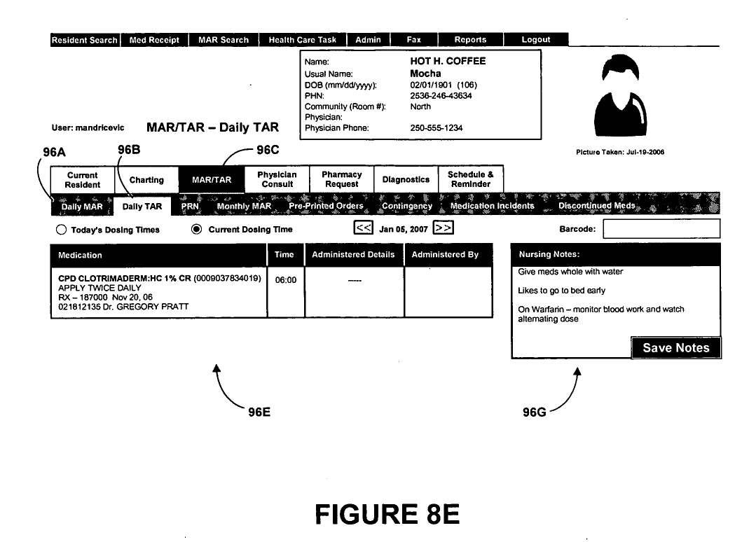

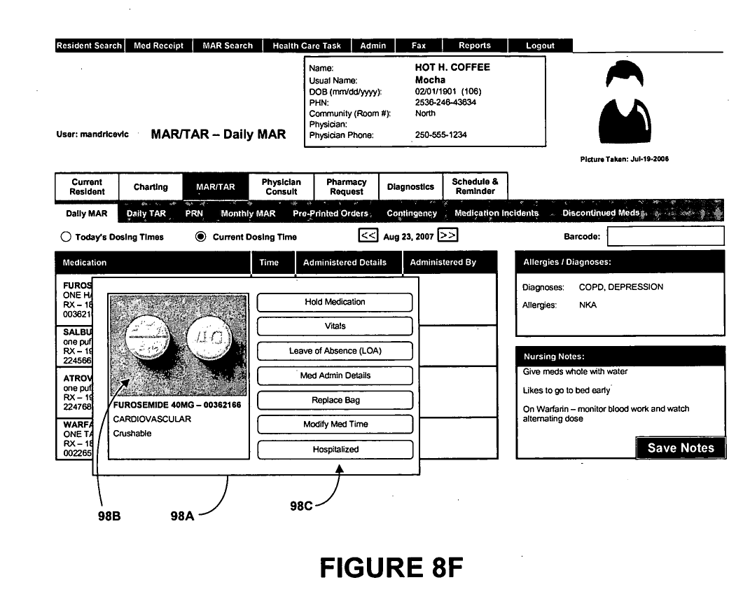

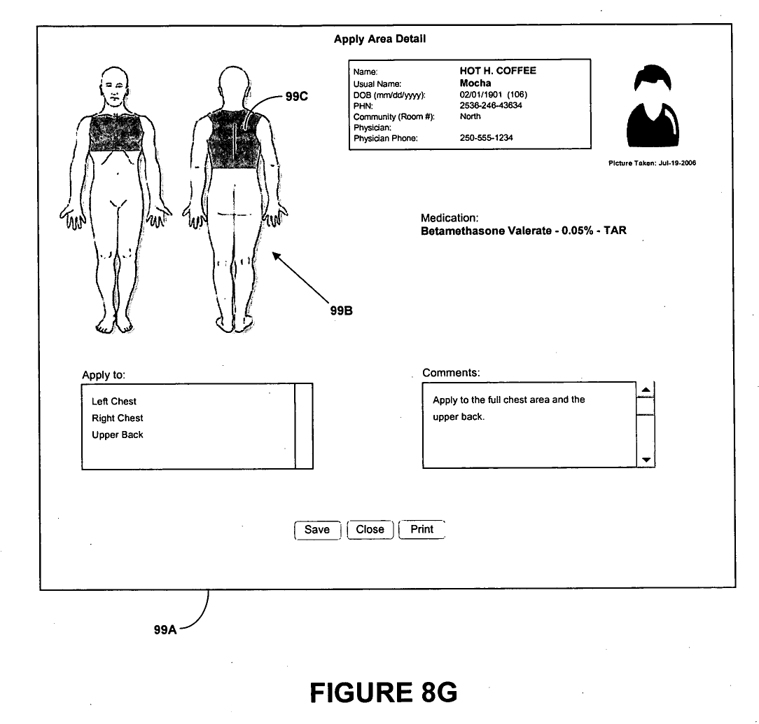

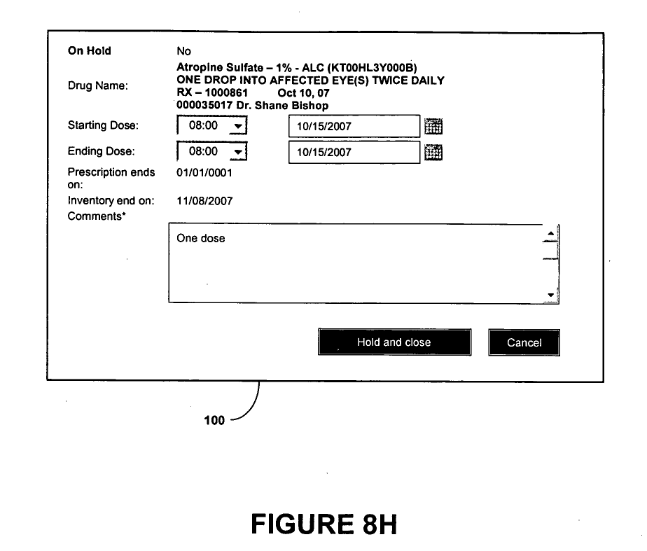

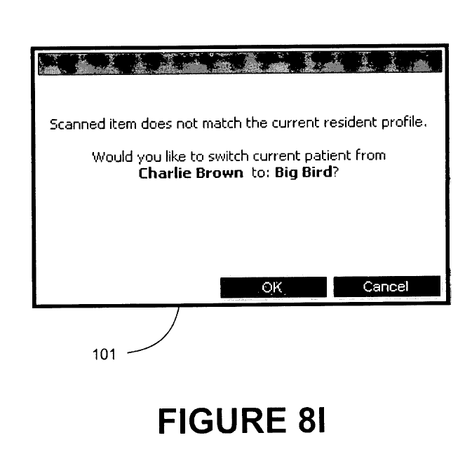

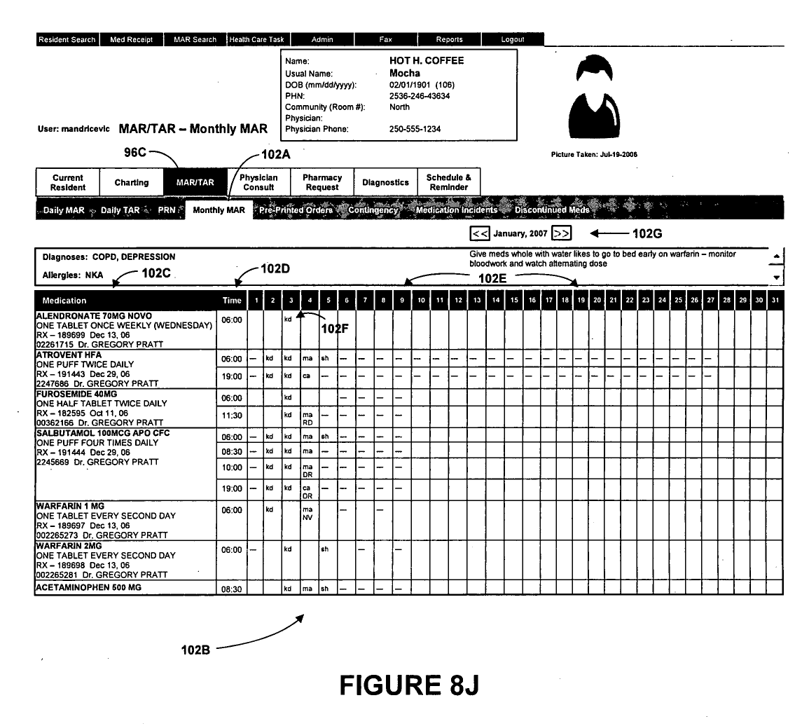

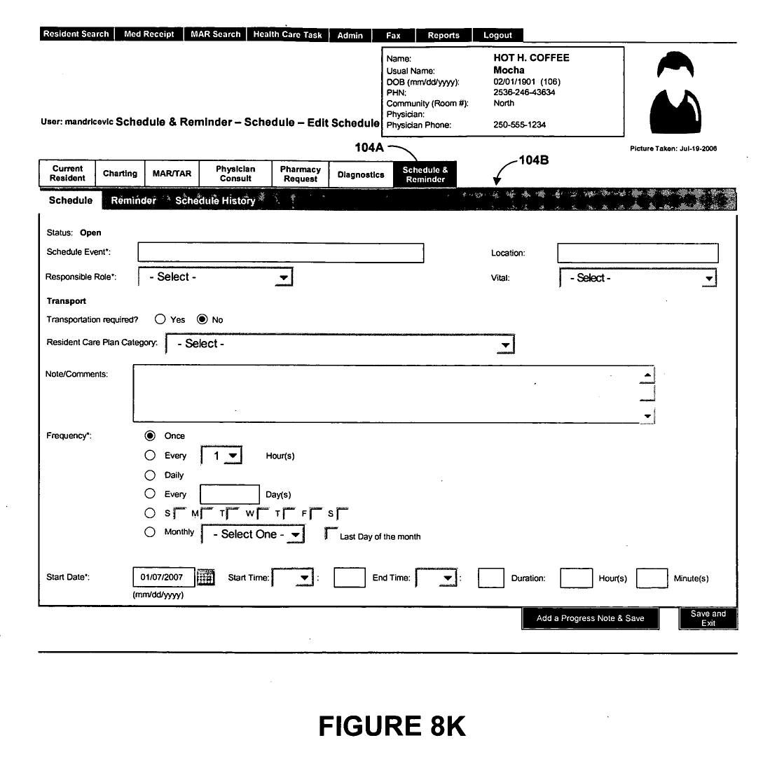

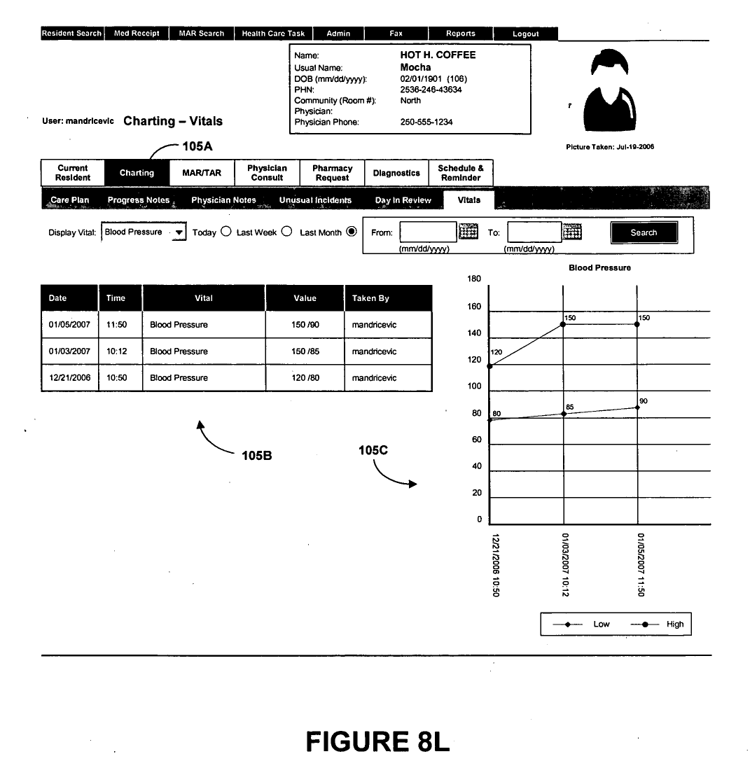

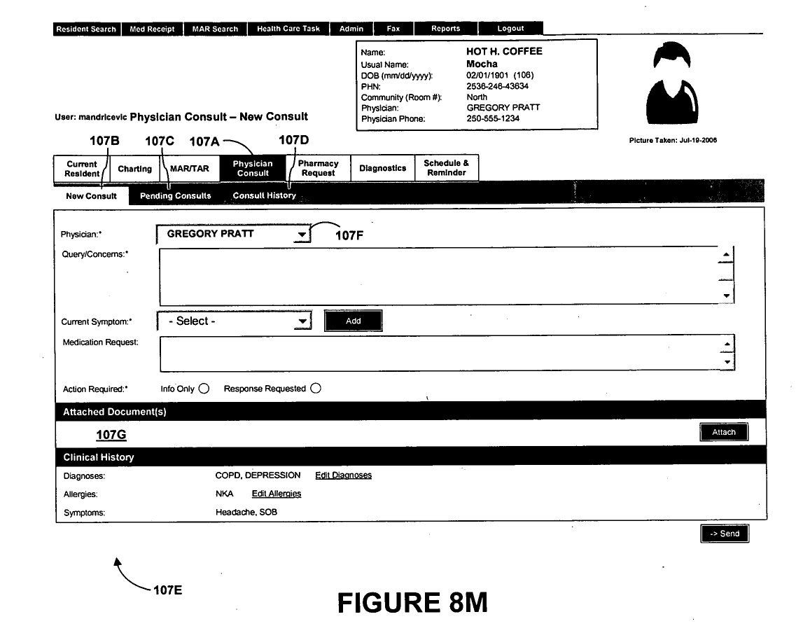

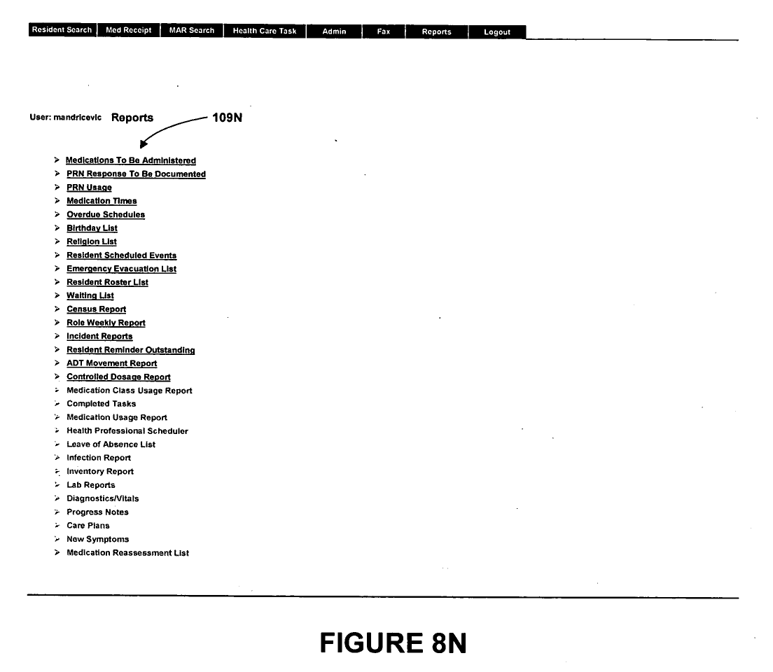

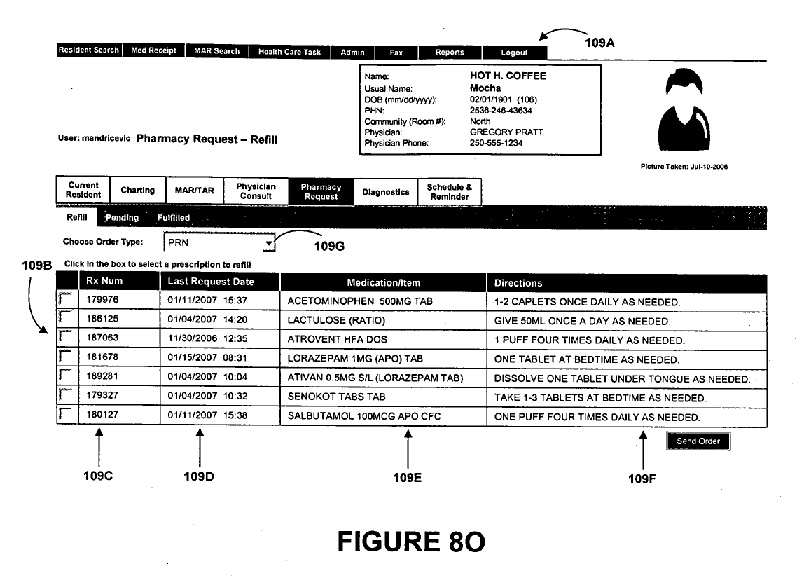

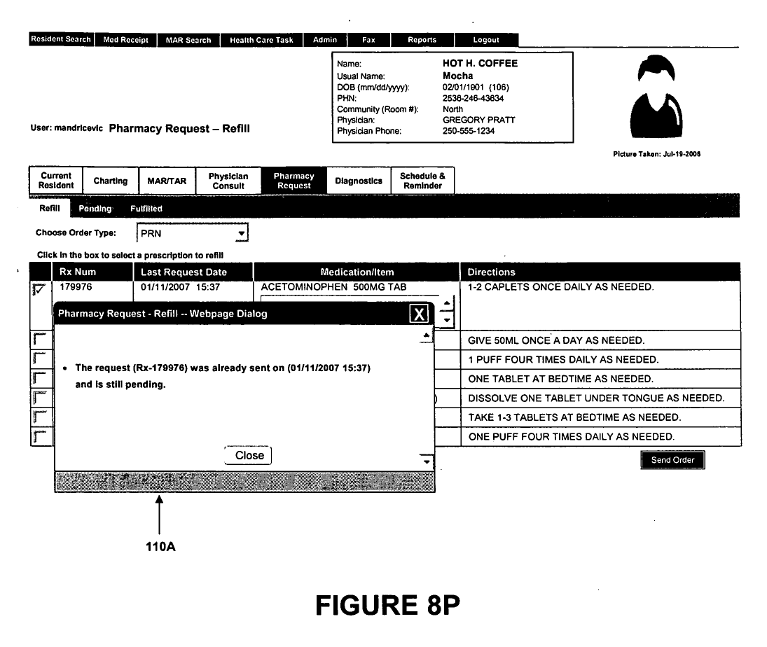

In carrying out administration of medications to a patient, the caregiver generally uses the eMAR component of the RMS. The eMAR includes a plurality of graphical user interface displays (e.g. web pages) through which the caregiver accesses the functionality of the eMAR and the data records contained in the RMS database. Interfaces of the exemplary embodiment are shown in FIGS. 8A-8P, but it is to be understood that such displays are illustrative only, since persons skilled in the art will be able to conceive of alternative interfaces for performing the functions described herein having regard to the present specification.

With reference to FIG. 8A, the RMS interface 90 includes a web portal having a number of functions which may be selected by the user to access the various features of the system. When a caregiver conducts their rounds, for each resident patient they must first identify the patient to whom medications are to be administered (step 73). As shown in FIG. 8A, a resident search page provides a number of options for identifying the patient. For example, the caregiver may manually enter the patient's name, in boxes provided for such purpose 91A. The page also provides boxes for searching by physician 91B, phone number 91C, community 91D, date of birth 91E, and status 91F. A list of available patients is also presented 91G, including some or all of the above-mentioned information, from which the correct patient may be selected.

Once the correct patient is selected, the caregiver may confirm the patient's identity by visually comparing the patient's face with a photograph presented through the system interface. Alternatively, the patient's identify may be confirmed by scanning a barcode embodying the unique patient ID on a wristband or other article on or with the patient.

In another alternative, scanning the patient barcode causes the system to bring up the patient's record for confirmation by the caregiver. In general, any means for confirming or entering patient identity may be employed.

A patient barcode may be printed on a wristband worn by the patient, and the scanning device may be a handheld device in wireless communication with the point-of-care workstation or RMS directly. Alternatively, the barcode may be printed on the patient's chart, or at any other suitable location near the patient. While a barcode embodying a unique patient ID is employed in the exemplary embodiment, any reliable means to identify patients may be employed, including a barcode or an embedded identification chip.

In yet another alternative, the patient's record is brought up for the caregiver to confirm the patient's identity when any medication package is scanned. The RMS database records related to the medication package identify the corresponding patient, whereby the patient's record is displayed for identity confirmation.

Once the patient's identity is entered into the workstation, and with reference to FIG. 8B, the patient record 95 for the identified resident is displayed (step 74). The homepage includes a number of tabs 95A for accessing various functions. Selection of a tab will generally cause a corresponding sub-page 95B to be displayed including any sub-tabs 95C for accessing the features of that sub-page. As shown in FIG. 8B, the “Current Resident” tab is selected and the “Homepage” sub-tab is selected. The resident homepage displays information regarding the resident, including an identification area 95D which shows identifying information (name, usual name, date of birth, photograph), patient phone number and community, and physician information including name and phone number. In a main area 95E of the page is displayed all of the relevant medical information, including areas showing: diagnoses, allergies, and symptoms 95F; scheduled events including medication administration events 95G; reminders 95H; progress notes 951; and vitals 95J. As shown in FIG. 8C, the page may further be configured to show, when first accessed, a pop-up dialogue box 95M showing reminders of medications due to be administered or any other desired reminders.

With reference to FIGS. 8D & 8E, administration of medication ordinarily proceeds by selecting the “Daily MAR” 96A and “Daily TAR” 96B sub-tabs of the “MAR/TAR” tab 96C. The “Daily MAR” sub-tab 96A accesses the daily MAR page 96D (for administration of medications having discrete and readily ascertainable dose measurements), while the “Daily TAR” sub-tab 96B access the corresponding daily TAR page (“topical administration record”, for administration usually of topical medications such as creams and ointments).

With reference to FIG. 8D, the Daily MAR displays, among other things, an area 96E listing doses of medication to be administered to the patient on the current dosing date 96R, and showing only such doses to be administered in the current dosing time, or all of today's dosing times, as may be selected 96S. The details for each of the scheduled doses are presented in a respective line item 96Q as shown in FIG. 8D. The information shown includes the medication name 96H, administration instructions 961, prescription number 96J and start date 96K, prescribing physician 96L, Drug Identification Number (DIN) 96V unique package ID 96M of the package containing the dose, and time for administration 96N. Once the dose is administered, any entered administration details 96O, and an identification of the caregiver who administered the dose 96P, are also shown. The page further contains an area 96F for entering and displaying pertinent medical information such as allergies/diagoses, and another area 96G for nursing notes such as administration instructions. Thus, the Daily MAR shows a complete record of all medication packages to be administered and other pertinent information.

The caregiver then proceeds to administer each package of medication. The caregiver begins by scanning a barcode on a medication package, or otherwise entering the unique package ID of the selected package (step 75). If the unique package ID of the package is included in the list of medication packages displayed in the Daily MAR (i.e. included in the medications scheduled to be administered at this date and time) (decision 76), the line item or items 96Q in the Daily MAR (or Daily TAR, as the case may be) corresponding to the scanned medication package is/are selected (e.g. highlighted green). The caregiver may then confirm that each selected dose is correct by comparing the information in the line item to the human-readable information imprinted on the package (step 77). If, for any reason, the package should not be administered, a message so indicating is displayed (step 83). As shown in FIG. 8F, the RMS database may also be configured to contain images for all or a predetermined subset of the medications contained in the database. Thus, when a caregiver scans a medication package, a dialogue box 98A showing an image 98B of the medication indicated in the selected line item may also be displayed on the workstation so that the caregiver may visually confirm that the medications contained in the scanned medication package are correct.

With further reference to FIG. 8F, the caregiver then opens the medication package and administers the medications according to the instructions printed on the medication package and displayed in the Daily MAR or a pop-up dialogue box shown when the package is scanned (step 78). The Daily MAR provides means (e.g. dialogue buttons 98C) for the caregiver to signify that the medication has been administered and also to record any further relevant information such as observations of the patient's vital signs, medication administration details such as incomplete administration of the medication (e.g. refusal by patient, medication is vomited), a replacement of the medication bag or a modification of the administration time, or the resident's absence due to a leave of absence or hospitalization. The caregiver may also indicate that the medication is on hold for any particular reason.

Since each medication package contains a single dose of at least one prescribed medication, and preferable a single dose of all of the medications prescribed for the date and time for administration of the package, when a package barcode is scanned all of the corresponding line items in the Daily MAR will be selected and highlighted, and administration of each dose proceeds as described above.

Furthermore, since the Daily MAR is directly connected to the RMS database, if any changes are made involving the medication from another source (e.g. a pharmacy), such changes are updated in real time in the Daily MAR displayed on the caregiver's workstation. For example, if, during medication administration, a particular medication is marked as discontinued by a pharmacy accessing the RMS database, then this indication will propagate to the Daily MAR in real time allowing the caregiver to make an informed decision as to how to handle the scheduled dose (e.g. replace the medication package, skip the dose, or administer the dose nevertheless).

Administration of topical medications proceeds essentially in the same manner as described above, except that the Daily TAR page shown in FIG. 8G is used and preferably also provides a graphic dialogue interface 99A depicting a representation the human body 99B wherein various regions of the body are defined thereby enabling the caregiver to indicate on what regions, e.g. 99C of the patient's body the topical medication was applied. It will be appreciated that the system may also be adapted to access such graphic dialogue interface from the Daily MAR page instead, as appropriate.

With individual-dose tracking of medications, any dose at any time can be put on hold. For example, if a physician decides to put every 06:00 dose on hold starting Wednesday and ending Friday, such adjustment can be entered into RMS easily without affecting or interfering with the other doses. In addition, the pharmacy which has filled the prescriptions may also put an entire prescription on hold for a fixed time period or indefinitely; this may be done at the pharmacy through the pharmacy's prescription management system which pushes the hold direction to the RMS. A dialogue box 100 illustrating this feature is shown in FIG. 8H.

The caregiver then proceeds to administer the next medication package in the same manner. Until all doses scheduled for the particular date and time have been administered (decision 79), the interface is locked on this particular patient's homepage (step 74) in order to ensure that the caregiver cannot accidentally proceed to the next patient in the round before completing administration of medications to this patient. Thus, if the caregiver attempts to move to the next patient before administering all scheduled doses for this patient, RMS reminds the caregiver (step 80) and the caregiver must proceed to administer the next scheduled medication package (step 75) or intentionally and explicitly interrupt administration to this patient (decision 81) and proceed to a different patient (step 82). A dialogue box 101 illustrating the reminder feature is shown in FIG. 8I.

With reference to FIG. 8J, the MAR/TAR tab 96C also provides a Monthly MAR sub-tab 102A which shows an eMAR which more closely resembles typical eMARs. This Monthly MAR shows a chart 102B listing, by row, all of the patient's medication prescriptions for a particular month 102G, with the leading column 102C of each row particularizing the medication with the same information as in the Daily MAR, as described above. Each row also contains a column for the scheduled dosing time 102D and a column for each date in the month 102E in which is recorded the electronic initials, e.g. 102F of the administering caregiver. The information in the Monthly MAR derives from the information recorded in the RMS database through the medication administration procedure described above (i.e. it is not entered directly into the chart), though in another embodiment the page could be configured so as to allow direct entry or correction of information. In this way, a complete and reliable record of medication administration for the month is instantly available.

The system also provides, and is extensible to provide, further functions additional to the above-described medication administration process. For example, and as shown in FIGS. 8K & 8L, tabs are provided for “Schedule & Reminder” 104A and “Charting” 105A. Selecting the first displays a page which enables the entry of an event schedule or reminder 104B. Selecting the second displays a page which shows a list of vital signs previously entered during medication administration occurrences (as described above) 105B, and presents the data in the form of a chart 105C for easy reference.

As shown in FIG. 8M, a “Physician Consult” tab 107A is provided to allow for the entry of information regarding a physician consult with the patient, including sub-tabs for entering new consults 107B, viewing pending consults 107C and consult history 107D. Key information such as clinical information 107E (including diagnoses, allergies, and symptoms) are displayed to facilitate the process. While the system defaults to the primary care physician, as indicated in the patient's record, any physician may be selected, e.g. from a drop-down box 107F. The page provides for the easy attachment of documents 107G, e.g. progress notes, incident reports, vitals. The interface thus supports informed decision-making by a physician, and the consultation record may be sent electronically to a nursing station and need not be printed and faxed as is typically done.

With reference to FIG. 8N, the system provides means to generate reports according to any parameters as are desired. The list of reports 109N shown in FIG. 8N is merely exemplary, and the system is extensible so as to be able to add new reports as needed.

With reference to FIG. 8D, the “PRN” 96T and “Contingency” 96U sub-tabs of the “MAR/TAR” tab 96C provide further medication administration recordal functions. A PRN (“pro re nata”) prescription is similar to an ordinary prescription but does not direct administration at particular times; instead, the medication is administered as needed. The PRN sub-tab provides an interface for the recordal of such administration. Similarly, the “Contingency” sub-tab provides for the recordal of the administration of medication on a contingency basis from the facility's contingency stock which is unassigned to any particular patient.

The system further provides an interface 109A, as shown in FIG. 8O, which enables the caregiver to order refills of the patient's prescriptions. A list of the patient's prescriptions 109B is shown, including prescription number 109C, last request date 109D, medication name 109E, and administration instructions 109F. A drop-down box 109G is provided enabling the caregiver to select between types of prescriptions (e.g. regular, PRN). The system is configured to ensure that erroneously duplicated refill requests are not transmitted to the pharmacy, by notifying the requestor (e.g. by a pop-up dialogue box 1110A, as shown in FIG. 8P) that a refill request has already been made if the RMS database indicates that such is the case.

### Inventory and Supply Chain Management

The exemplary system provides for improved inventory and supply chain management of medications, especially in respect of a long-term health care facility or prison having numerous resident patients.

At the beginning of the supply chain, uniquely identifiable individual-dose medication packages are produced by an automated packager as described above. In addition, prepackaged medications are assigned unique package IDs and labels bearing such ID are applied. Each such package may then be scanned by the pharmacy and recorded by the filter application for transmission to the RMS database.

The medication is delivered to the facility and items arriving at the facility are scanned into and registered with the facility's RMS. The medication packages are then stored pending administration in accordance with the facility's preferred practice. For example, the medications packages for a specific patient may be placed by facility staff into resident specific bins.

Finally, at the point of care, each medication package is scanned to verify correct dose, medication, and time of administration. Since every dose of medication is tracked from packaging to administration, inventory management is straightforward.

The system further enables the tracking of wasted or destroyed medications, and any medications returned to the pharmacy for any reason.

RMS may further be adapted to interface with the pharmacy order system to submit refill requests of existing prescriptions. The feature allows the facility to set, in respect of any particular prescription, a minimum desired inventory for the corresponding medication. Once the regular administration of medication packages causes a reduction of the medication's inventory below the minimum inventory level, the system may be configured to automatically interface with the pharmacy management system in order to submit a request for refill. Such feature assists in the maintenance of appropriate inventory levels, thereby reducing the frequency of emergency fill requests, as well as reducing paper usage.

In addition, and as indicated above, upon intercepting a prescription fill or refill request, the filter application may be configured to check for existing inventory at the facility in respect of the medications specified in the prescription fill or refill request, as the case may be. For example, a patient's prescription for single doses of medication is replaced with a new prescription for double doses of the same medication. The filter application checks the RMS database to determine if any of the single-dose medication packages remain in inventory and, if so, provides for the filtering off of such doses from the packaging instructions so that the automated packager will produce only the needed additional doses. The filter application may further be configured to update the RMS database records corresponding to the single-dose medication packages already in inventory to be associated with the new prescription. In other words, the filter application ‘transfers’ the existing inventory of single doses of the medication from the discontinued prescription to the new prescription. In this way, there is no need to return medication packages already in inventory, and the eMAR accurately reflects the prescription underlying the medication doses when administered. Such is not possible in known systems which do not uniquely track medications at the level of single doses.

Similarly, in situations where a particular medication's Drug Identification Number (DIN) changes for any reason, and such change is updated in association with a prescription underlying the medication, the present system easily provides for updating such association in connection with every single uniquely-identifiable and trackable dose of affected medication.

### Lot and Expiry Date Tracking

Tracking lot numbers or expiry dates is impractical in a manual system. While some automated packager systems track lot numbers when packaging medication, the tracking information is not integrated into the facility's administration management or inventory systems. Accordingly, in known systems it is not feasible to track medication lot numbers and expiry dates throughout the complete supply chain from packaging to point of care.

The invention provides lot number and expiry date tracking for each dose of medication throughout the supply chain. Each medication package containing doses of multiple medications is uniquely identifiable as soon it is created. The RMS database records corresponding to the medication package contains all the relevant information about the contained medications, including the lot numbers and expiry dates of the medications. Therefore, the lot number and expiry date of every dose of medication which has ever been packaged and administered or is remaining in inventory is accessible throughout the entire supply chain right up to the point of care.

Many advantages flow from the traceability of unit doses of medication. The system automatically provides security against the proliferation of counterfeit drugs, as counterfeit drugs would not be packaged with the correct unique package ID and package contents. Furthermore, each dose can be traced back to the place of manufacture along with material compositions and test results for the lot. Likewise, every unit dose of medication could be traced to its ultimate destination in the event of a recall.

In respect of expiry dates, known medication packages generally bear the expiry or best before date of it contents, but a caregiver might not check the expiry date before administering the medication. Since each unit dose of medication is tracked in the exemplary system and is associated in the RMS database to the expiry date of the medication, the eMAR will automatically alert the caregiver that the medication has expired once the caregiver scans the package. The caregiver may then elect to administer the medication nevertheless or may take some other action such as returning the medication to the pharmacy or destroying the medication locally. In any event, no expired medication may be administered without the caregiver's knowledge.

Systems employing automated packagers without tracking on an individual-dose basis remain susceptible to errors, e.g. when medication for a particular dosing time is missing or when medications are given at the wrong time. Furthermore, without tracking each individual-dose package on a patient-by-patient basis, it is still possible to administer the right medications at the right time, but to the wrong patient. By uniquely identifying each individual-dose medication package, the above-described system is capable of notifying the caregiver if any of the medication, time, or patient are incorrect for an proposed administration.

Although various exemplary embodiments of the invention have been disclosed, it will be apparent to persons skilled in the art that various changes and modifications can be made which will achieve the advantages of the invention without departing from the true scope of the invention.

For example, although the exemplary system has been described as cooperating with a single pharmacy prescription management system, a single automated packager, a single inventory workstation, and a single point-of-care workstation, persons skilled in the art will appreciate that the system can cooperate with any number of these items. As illustrated in FIG. 6, the RMS may interface with a plurality of pharmacies, each of which may employ a plurality of automated packagers of various types and configurations. Furthermore, a plurality of filter application modules cooperating with a plurality of pharmacy systems and automated packagers may cooperate with the RMS. Lastly, a typical facility will have a number of point-of-care workstations in use, often simultaneously. Generally, persons skilled in the art may implement the invention in accordance with the particular needs at hand and remain within the scope and spirit of the invention. Furthermore, persons skilled in the art will appreciate that the various systems and devices described herein may be implemented in any number of forms more or less suitable to the particular situation.

It is further to be appreciated that, in some embodiments, the RMS and/or filter application are external to any particular facility and are accessible to, and provide the herein-described services and functionality, to a plurality of facilities at once. In one such embodiment, the RMS resides on one or more remote servers and interfaces with pluralities of filter application modules, point-of-care workstations, and inventory workstations via a network connection such as the Internet. In such embodiments, the network connection between the various systems is over a dedicated private network or over a public network, but in any event is preferably a secure connection employing, for example, HTTPS and/or VPN technology. Furthermore, in such embodiments the RMS securely stores and maintains information so as to restrict access to the corresponding facility.

It will further be appreciated by persons skilled in the art that the various cooperating systems described herein may, in other embodiments, be implemented in a single system wherein the various services and functionalities are performed by cooperating modules within the single system. Furthermore, it will be appreciated that the functionality described as being performed by one cooperating system may instead be performed by another cooperating system without necessarily departing from the scope or spirit of the invention. In particular, it will be appreciated that a specific functionality of a described system may instead be performed by a further subsystem operatively connected to the first system. For example, the RMS database may instead reside on a separate, dedicated database server operatively connected via a network or otherwise to the system operating the RMS eMAR.

Embodiments of the invention may be implemented in any conventional computer programming language. For example, preferred embodiments may be implemented in a procedural programming language (e.g. “C”) or an object oriented language (e.g. “C++”). Alternative embodiments of the invention may be implemented as pre-programmed hardware elements, other related components, or as a combination of hardware and software components.

Embodiments can be implemented as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server over the network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

It is to be appreciated that the section headings appearing hereinbefore do not limit the scope of the invention as described but are merely intended to organize the description for the sake of clarity.

With the foregoing exemplary embodiments having been disclosed, it will be apparent to those skilled in the art that various changes and modifications can be made to appropriately suit the needs and objectives of another application and still achieve the advantages of the invention; all such changes and modifications are intended to fall within the scope of the invention as defined by the claims that follow.

We claim:

1. A system for managing administration of prescribed medications to patients, the system comprising:
a server for receiving prescription fill requests which are based on prescriptions for the prescribed medications, for causing uniquely identifiable medication packages to be produced based on the prescription fill requests and in conformity with the prescriptions, each medication package containing a single dose of at least one of the prescribed medications, and for receiving and maintaining medication package records specifying the contents of each particular medication package; and
an interface for providing access to the prescriptions and the medication package records for validating administration of the prescribed medications to the patients
wherein each single dose of prescribed medication is uniquely identifiable by identifying the medication package containing that single dose and by accessing the medication package records specifying the contents of that medication package.

2. The system according to claim 1 wherein each medication package is uniquely associated with a corresponding unique package ID and is uniquely identifiable by that unique package ID.

3. The system according to claim 2 wherein causing uniquely identifiable medication packages to be produced comprises causing, for each medication package, a barcode uniquely encoding the unique package ID of that medication package to be imprinted on that medication package, and wherein identifying that medication package comprises reading the barcode imprinted on that medication package.

4. The system according to claim 3 wherein the medication packages are produced by an automated medication packager, and wherein, for each medication package, the barcode encoding the unique package ID of that medication package is imprinted on that medication package by the automated medication packager.

5. The system according to claim 4 wherein the unique package ID of each medication package is generated by the automated medication packager.

6. The system according to claim 5 wherein the medication package records are received from the automated medication packager.

7. The system according to claim 6 wherein each unique package ID comprises a barcode header and a bag ID, wherein the barcode header uniquely identifies a corresponding plurality of medication packages associated with a corresponding specific one of the patients.

8. The system according to claim 2 wherein causing uniquely identifiable medication packages to be produced based on the prescription fill requests and in conformity with the prescriptions comprises filtering from each prescription fill request doses of prescribed medication exceeding the prescriptions.

9. The system according to claim 8 wherein specifying the contents of any particular medication package includes specifying:
for each single dose of prescribed medication contained in that medication package:
a size of the single dose,
an identification of the prescribed medication, and
administration instructions; and
for all of the prescribed medications contained in that medication package:
a specific one of the patients, and
an administration date and time.

10. The system according to claim 9 wherein specifying the contents of any particular medication package further includes specifying, for each single dose of prescribed medication contained in that medication package, an expiry date and a lot number.

11. A system for tracking a dose of a medication and for validating an administration of the dose of the medication to a patient, the system comprising:
a module for receiving prescription information and a data record, each associated with the dose of the medication, the data record being further associated with a unique package ID uniquely identifying a medication package containing the dose of the medication and no other dose of the medication;
a database for receiving and storing the data record in association with the unique package ID, and for receiving and storing the prescription information; and
an interface for providing access to the database for accessing the data record and the prescription information for validating the administration of the dose of the medication to the patient, wherein the data record is accessed by reference to the unique package ID.

12. The system according to claim 11 wherein the module is further for conforming a prescription fill request to the prescription information thereby creating a filtered fill request, the prescription fill request being based on the prescription information, and for causing the medication package to be produced in accordance with the filtered fill request.

13. The system according to claim 12 wherein conforming the prescription fill request to the prescription information comprises filtering off a dose of another medication which exceeds a prescription period of the prescription information.

14. The system according to claim 12 wherein causing the medication package to be produced in accordance with the filtered fill request comprises transmitting the filtered fill request to an automated medication packager thereby causing the automated medication packager to produce the medication package, to generate the unique package ID and the data record, and to transmit the data record in association with the unique package ID to the module.

15. The system according to claim 14 wherein transmitting the filtered fill request to the automated medication packager further causes the automated medication packager to imprint on the medication package a barcode uniquely encoding the unique package ID.

16. The system according to claim 15 wherein transmitting the filtered fill request to the automated medication packager further causes the automated medication packager to imprint on the medication package human-readable information based on the prescription information and the data record.

17. The system according to claim 16 wherein the medication package further contains a dose of at least one other medication.

18. The system according to claim 16 wherein accessing the data record by reference to the unique package ID comprises entering the unique package ID into the interface.

19. The system according to claim 18 wherein entering the unique package ID into the interface comprises using a barcode reader to read the barcode imprinted on the medication package, the barcode encoding the unique package ID.

20. The system according to claim 19 wherein validating the administration of the dose of the medication to the patient comprises comparing the data record and the prescription information.

21. The system according to claim 20 wherein the data record is further uniquely associated with the patient, and validating the administration of the dose of the medication to the patient further comprises identifying the patient.

22. The system according to claim 20 wherein the interface is further for displaying at least a portion of each of the data record and the prescription information, and wherein validating the administration of the dose of the medication to the patient further comprises comparing the human-readable information imprinted on the medication package with the at least a portion of the data record or the at least a portion of the prescription information.

23. A system for producing a uniquely identifiable medication package containing a single dose of a prescribed medication, the system comprising:
an interface for receiving a prescription fill request which is based on a prescription for the prescribed medication;
a processor for conforming the prescription fill request to the prescription thereby producing a filtered fill request, and for causing the medication package to be produced in accordance with the filtered fill request, wherein the medication package is uniquely identifiable by a unique package ID;
an interface for receiving data regarding the single dose of the prescribed medication, the data being associated with the unique package ID of the medication package; and
a database for storing the data.

24. A method for uniquely identifying a single dose of a prescribed medication for validating an administration of the single dose to a patient, the method comprising:
receiving a prescription fill request which is based on a prescription for the prescribed medication;
conforming the prescription fill request to the prescription thereby producing a filtered fill request;
causing a uniquely identifiable medication package to be produced in accordance with the filtered fill request, the medication package containing, with respect to the prescribed medication, exclusively the single dose of the prescribed medication, wherein the medication package is uniquely identifiable by a unique package ID;
receiving data regarding the single dose, the data being associated with the unique package ID of the medication package;
storing the prescription and the data, the data being stored in association with the unique package ID;
providing access to the prescription and the data, wherein the data is accessed by reference to the unique package ID, and whereby the single dose of prescribed medication is uniquely identified, for validating the administration of the single dose to the patient.

25. A computer program product for enabling a computer to uniquely identify a single dose of a prescribed medication for validating an administration of the single dose to a patient, the computer program product comprising:
software instructions for enabling the computer to perform predetermined operations; and
a computer readable medium bearing the software instructions,
the predetermined operations including the steps of:
receiving a prescription fill request which is based on a prescription for the prescribed medication;
conforming the prescription fill request to the prescription thereby producing a filtered fill request;
causing a uniquely identifiable medication package to be produced in accordance with the filtered fill request, the medication package containing, with respect to the prescribed medication, exclusively the single dose of the prescribed medication, wherein the medication package is uniquely identifiable by a unique package ID;
receiving data regarding the single dose, the data being associated with the unique package ID of the medication package;
storing the prescription and the data, the data being stored in association with the unique package ID;
providing access to the prescription and the data, wherein the data is accessed by reference to the unique package ID, and whereby the single dose of prescribed medication is uniquely identified, for validating the administration of the single dose to the patient.
